(19) United States
(12) Patent Application Publication
(10) Pub. No.: US 2024/0293651 A1
(43) Pub. Date: Sep. 5, 2024

(54) CAPSULE ADMINISTERING ASSEMBLY

(71) Applicant: Novo Nordisk A/S, Bagsvaerd (DK)

(72) Inventors: Nikolaj Eusebius Jakobsen, Soeborg (DK); Kuldeep Sanger, Kurukshetra (IN)

(21) Appl. No.: 18/573,470

(22) PCT Filed: Jul. 9, 2021
(86) PCT No.: PCT/EP2021/069089

§ 371 Date: Dec. 22, 2023

(51) Int. Cl. A61M 31/00

(52) U.S. Cl. CPC A61M 31/002; A61M 2205/106; A61M 2210/1042

(57) ABSTRACT

A capsule administering assembly (100, 100′) comprising an ingestible capsule (200, 200′) for delivery of a mixed drug product to a subject user, and a capsule accessory device (300, 300′), wherein the ingestible capsule is operable from a first attached state where the ingestible capsule (200, 200′) is coupled relative to the capsule accessory device (300, 300′), and a second detached state wherein the ingestible capsule (200, 200′) is detached from the capsule accessory device (300, 300′) to permit ingestion of the ingestible capsule. The capsule administering assembly (100, 100′) comprises a first reservoir (231) holding a first drug component (A) and a second reservoir (332, 362), the second reservoir (332, 362) holding a second drug component (B).

The present invention relates to ingestible devices for delivery of a mixed drug product to a subject user and assemblies for preparing a mixed drug product within the ingestible device.

### BACKGROUND OF THE INVENTION

In the disclosure of the present invention reference is mostly made to the treatment of diabetes by delivery of insulin, however, this is only an exemplary use of the present invention.

May people suffer from diseases, such as diabetes, which requires them to receive injections of drugs on a regular and often daily basis. To treat their disease these people are required to perform different tasks which may be considered complicated and may be experienced as uncomfortable. Furthermore, it requires them to bring injection devices, needles and drugs with them when they leave home. It would therefore be considered a significant improvement of the treatment of such diseases if treatment could be based on oral intake of tablets or capsules.

However, such solutions are very difficult to realise since protein-based drugs will typically be degraded and digested rather than absorbed when ingested.

To provide a working solution for delivering insulin into the bloodstream through oral intake, the drug has to be delivered firstly into a lumen of the gastrointestinal tract and further into the wall of the gastrointestinal tract (lumen wall). This presents several challenges among which are: (1) The drug has to be protected from degradation or digestion by the acid in the stomach. (2) The drug has to be released while being in the stomach, or in the lower gastrointestinal tract, i.e. after the stomach, which limits the window of opportunity for drug release. (3) The drug has to be delivered at the lumen wall to limit the time exposed to the degrading environment of the fluids in the stomach and in the lower gastrointestinal tract. If not released at the wall, the drug may be degraded during its travel from point of release to the wall or may pass through the lower gastrointestinal tract without being absorbed, unless being protected against the decomposing fluids.

Prior art references relating to delivery of oral dosing of reconstituted active agents include US 2004253304 A1 and US2314088486 A1. These references suggest forming the reconstituted agents at the site of the target location, i.e. in-situ.

Having regard to the above, it is an object of the present invention to provide an ingestible device for swallowing into a lumen of a gastrointestinal tract, and which allows a mixed drug product to be prepared in a less complex manner. A further object of the present invention is to provide an ingestible device suitable for delivering a mixed liquid drug product which enable a high drug load and offer improvements with regard to storage and drug delivery.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention, a capsule administering assembly is provided comprising an ingestible capsule for delivery of a mixed drug product to a subject user, and a capsule accessory device. The ingestible capsule is operable from a first attached state where the ingestible capsule is coupled relative to the capsule accessory device, and a second detached state wherein the ingestible capsule is detached from the capsule accessory device to permit ingestion of the ingestible capsule. The capsule administering assembly comprises a first reservoir holding a first drug component (A) and a second reservoir separate from the first reservoir, the second reservoir holding a second drug component (B), and wherein the capsule administering assembly further comprises fluid communication means for establishing fluid communication between the first reservoir and the second reservoir.

The ingestible capsule comprises:
a capsule housing sized to pass through the gastrointestinal tract of a patient, the capsule housing comprising a capsule housing first portion,
the first reservoir arranged within the capsule housing,
a drug outlet, and
an expelling assembly comprising a movable wall associated with the first reservoir, the movable wall being movable relative to the capsule housing first portion to reduce the volume of the first reservoir thereby expelling drug from the first reservoir through the drug outlet.

The capsule accessory device comprises a first handling component configured for movement relative to the capsule housing first portion, wherein when the ingestible capsule assumes the first attached state, the first handling component is moveable relative to the capsule housing first portion from a first position to a second position, wherein the capsule administering assembly is configured to transfer the second drug component (B) from the second reservoir via the fluid communication means to the first reservoir upon movement of the first handling component from the first position to the second position, thereby forming said mixed drug product accommodated in the first reservoir.

In accordance with the first aspect, improved drug stability is offered compared to prior art systems. In addition, increased drug load is enabled. On the other hand, the invention according to the first aspect offers increased use of the available volume of the ingestible capsule, enabling the option of reducing the overall volume of the ingestible capsule, or improving the drug expelling performance.

In some forms the capsule accessory device comprises said second reservoir. In other embodiments, the ingestible capsule comprises both the first reservoir and the second reservoir.

In some forms, the capsule administering assembly is configured to increase the volume of the first reservoir upon movement of the first handling component from the first position to the second position thereby forcing the second drug component (B) from the second reservoir to the first reservoir. In such embodiments, the second drug component (B) may be sucked into the first reservoir for mixing the drug components.

In some embodiments the movable wall comprises a piston slidably arranged within a cylinder portion defined by the first reservoir. In other embodiments, the first reservoir may comprises a flexible wall which is configured to collapse for reducing the volume of the first reservoir.

In some embodiments, the capsule administering assembly is configured to reduce the volume of the second reservoir upon movement of the first handling component from the first position to the second position thereby forcing the second drug component (B) from the second reservoir to the first reservoir.

In some forms, the capsule accessory device further comprises a second handling component being movably arranged relative to the first handling component, wherein when the ingestible capsule assumes the attached state, the first handling component moves relative to the second handling component as the first handling component moves relative to the capsule housing first portion from said first position to said second position.

In such forms, in particular embodiments, the first handling component may be configured for rotationally movement relative to the capsule housing first portion from the first position to the second position, such as a rotational only movement without axial relative movement or, alternatively, with a combined rotational and axial relative movement.

In such embodiments, the capsule housing may be provided in a form where it further comprises a capsule housing second portion that is rotationally movable relative to the capsule housing first portion.

Also, in such embodiments, the administering assembly may be so configured that, as the first handling component moves relative to the capsule housing first portion from said first position to said second position, the first handling component couples rotationally with the capsule housing second portion and the second handling component couples rotationally with the capsule housing first portion.

In further embodiments of the capsule administering assembly, wherein the movable wall defines comprises a displaceable piston, and wherein the ingestible capsule comprises a mechanism coupling the displaceable piston, the capsule housing first portion and the capsule housing second portion with each other, wherein said mechanism incorporates a helical guide structure configured to displace the piston to increase the volume of the first reservoir as the capsule housing second portion rotates relative to the capsule housing first portion.

when the ingestible capsule assumes the attached state, the first handling component and the second handling component in combination define a cavity, wherein the ingestible capsule is partly of fully received in the cavity.

The first handling component and the second handling component may be configured to become mechanically separated relative to each other so as to remove the ingestible capsule from the cavity. Alternatively, the first handling component or the second handling component may provide an opening which enables the ingestible capsule accommodating the mixed drug product to be removed through the opening.

The capsule administering assembly may be formed to comprise a guide system enabling the ingestible capsule to be detached relative to the capsule accessory device subsequently to the first handling component being moved relative to the capsule housing first portion from the first position to the second position, but wherein the guide system prevents detachment relative to the capsule accessory device prior to the first handling component being moved to the second position. This prevents unintended use of the assembly.

In further embodiments the first handling component is configured for axially moving relative to the capsule housing first portion from the first position to the second position. In embodiments where first and second handling components are provided the axial movement may occur by an axial telescopic movement, e.g. by moving the first and second handling components axially towards each other.

In some forms of the capsule administering assembly, the fluid communication means comprises a fluid gate operable between a closed state wherein the fluid gate separates the first drug component (A) and the second drug component (B) from each other, and an open state wherein the fluid gate enables fluid flow from the second reservoir to the first reservoir.

In some variants the fluid communication means comprises said drug outlet. In alternative variants, the fluid communication means are formed as a drug inlet separately from said drug outlet.

In some embodiments one of the first drug component (A) and the second drug component (B) is a powder, and wherein the other of the first drug component (A) and the second drug component (B) is a liquid. In some forms wherein the drug component is provided as a powder, the powder may, prior to mixing, be accommodated in the first reservoir.

In other embodiments the first drug component (A) is a liquid and the second drug component (B) is a liquid.

The ingestible capsule may be provided so as to comprise an expelling assembly that comprises an energy source configured for exerting a load on the movable wall. Further, it may comprise a releasable trigger configured for actuation in response to one or more predetermined conditions to permit said load to move the movable wall thereby reducing the volume of the first reservoir and expelling said mixed drug product through the drug outlet.

The expelling assembly may be provided so that it is configured for accumulation of potential energy in the energy source in response to movement of the first handling component relative to at least one portion of the capsule housing and/or the second handling component. In such system, reduced risk of creep of plastic components is to be expected offering improved storage capability.

In some embodiments, the energy source is or comprises at least one spring configured as a drive spring. Exemplary springs include a compression spring, a torsion spring, a leaf spring or a constant-force spring. The spring may either be strained or configured for being strained for powering expulsion from the capsule device. Other non-limiting exemplary types of energy sources for the actuator include compressed gas actuators or gas generators. In some embodiments, in the pre-firing configuration, the energy source exerts a load onto the movable wall thereby biasing the movable wall for reducing the volume of the first reservoir. In other embodiments the energy source is configured to exert a load onto the movable wall only upon triggering of a trigger member or mechanism of the capsule device.

The capsule may comprise one or more openings to allow a biologic fluid, such as gastric fluid, to enter the capsule for dissolving the dissolvable firing member(s).

According to exemplary embodiments described herein, a trigger of a capsule device may be configured to actuate the capsule device in the GI tract of a subject, or in any other location internal to a subject, under a predetermined condition. In some embodiments, the predetermined condition includes one or more of a predetermined time after ingestion of the capsule device, a predetermined location in the GI tract, physical contact with the GI tract, physical manipulation in the GI tract (e.g., compression via peristalsis), one or more characteristics of the GI tract (e.g., pH, pressure, acidity, temperature, etc.), or combinations thereof. In some embodiments, the trigger may be a passive component configured to interact with the environment of the GI tract to actuate the capsule device.

In some embodiments, the trigger may be a sensor that detects one or more characteristics of the GI tract. For example, a sensor detecting contact with a GI mucosal lining may be used to actuate the device. In embodiments where a sensor is employed, the trigger may also include an active component that moves in response to a predetermined condition being detected by the sensor. For example, a gate may be moved when contact with a GI mucosal tract is detected. In other embodiments the trigger may employ electrical power to melt or weaken a rupturable membrane (e.g., by applying a voltage across a conductive rupturable membrane) and/or trigger a chemical reaction. Of course, any suitable active or passive trigger may be employed for a capsule device, as the present disclosure is not so limited.

In some forms the drug outlet comprises a nozzle arrangement configured for needleless jet delivery. The nozzle arrangement may be provided with one jet nozzle, or alternatively a plurality of jet nozzles.

In other forms, the drug outlet may be provided so that it comprises an injection needle, or a plurality of injection needles.

In exemplary embodiments, the capsule device is configured for swallowing by a patient and travelling into a lumen of a gastrointestinal tract of a patient, such as the stomach, the small intestine or the large intestine. The capsule of the device may be shaped and sized to allow it to be swallowed by a subject, such as a human.

By the above arrangements an orally administered mixed drug substance can be delivered safely and reliably into the stomach wall or intestinal wall of a living mammal subject.

As used herein, the terms “drug”, “drug substance” or “drug product” is meant to encompass any drug formulation capable of being delivered into or onto the specified target site. The drug may be a single drug compound or a premixed or co-formulated multiple drug compound. Representative drugs include pharmaceuticals such as peptides (e.g. insulins, insulin containing drugs, GLP-1 containing drugs as well as derivatives thereof), proteins, and hormones, biologically derived or active agents, hormonal and gene-based agents, nutritional formulas and other substances in both solid, powder or liquid form. Specifically, the drug may be an insulin or a GLP-1 containing drug, this including analogues thereof as well as combinations with one or more other drugs.

### BRIEF DESCRIPTION OF THE DRAWINGS

In the following embodiments of the invention will be described with reference to the drawings, wherein

FIG. 1 is an external perspective view of an ingestible capsule 200 of a capsule administering assembly 100 according to a first embodiment of the invention,

FIG. 2a is an external perspective view of capsule accessory device 300 of the capsule administering assembly 100 according to the first embodiment of the invention,

FIG. 2b is an external perspective partly cut view of the capsule administering assembly 100 comprising ingestible capsule 200 accommodated within capsule accessory device 300,

FIG. 3 is an exploded perspective view of components of the capsule administering assembly 100 according to the first embodiment of the invention,

FIG. 4a is a perspective view of ingestible capsule 200,

FIG. 4b is a perspective partly cut view of ingestible capsule 200,

FIG. 5a is a perspective view of a needle hub 250, dissolvable firing member 280 and injection needle 290 in accordance with the first embodiment,

FIG. 5b is a perspective view of components of FIG. 5a omitting the dissolvable firing member 280 and injection needle 290 from view,

FIGS. 5c and 5d show a perspective view and a partly cut perspective view of a middle housing portion 220 of ingestible capsule 200,

FIGS. 6a, 6b and 6c show respectively a perspective view, a perspective partly cut view and a cross-sectional side view of ingestible capsule 200 in a ready-to-ingest state,

FIG. 7 is a perspective partly cut view of capsule accessory device 300 omitting the ingestible capsule 200 from view,

FIG. 8a is a cross-sectional side view of capsule administering assembly 100 in a state prior to drug mixing,

FIG. 8b is a cross-sectional side view of capsule administering assembly 100 in a state after drug mixing,

FIGS. 9a through 9d show a sequence of perspective views of first and second handling components 310 and 330 of capsule accessory device 300 in different states during operation,

FIG. 10 is an exploded perspective view of components of the capsule administering assembly 100′ according to a second embodiment of the invention,

FIGS. 11a and 11b are cross-sectional side views of the second embodiment capsule administering assembly 100′ in a state prior to drug mixing, rotated relative to each other by 90°,

FIGS. 12a and 12b are cross-sectional side views of capsule administering assembly 100′ in a state after drug mixing, rotated relative to each other by 90°,

FIGS. 13a and 13b are cross-sectional side views of an ingestible capsule 200′ of the second embodiment capsule administering assembly 100′ in a state prior to drug mixing, rotated relative to each other by 90°,

FIGS. 14a and 14b are cross-sectional side views of the ingestible capsule 200′ of the second embodiment in a state after drug mixing, rotated relative to each other by 90°,

FIGS. 15a and 15b are cross-sectional side views of the ingestible capsule 200′ of the second embodiment in a ready-to-ingest state, rotated relative to each other by 90°, and

FIGS. 16a and 16b are cross-sectional side views of the ingestible capsule 200′ of the second embodiment in a state after drug expelling, rotated relative to each other by 90°.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The terms “assembly” and “subassembly” do not imply that the described components necessarily can be assembled to provide a unitary or functional assembly or subassembly during a given assembly procedure but is merely used to describe components grouped together as being functionally more closely related.

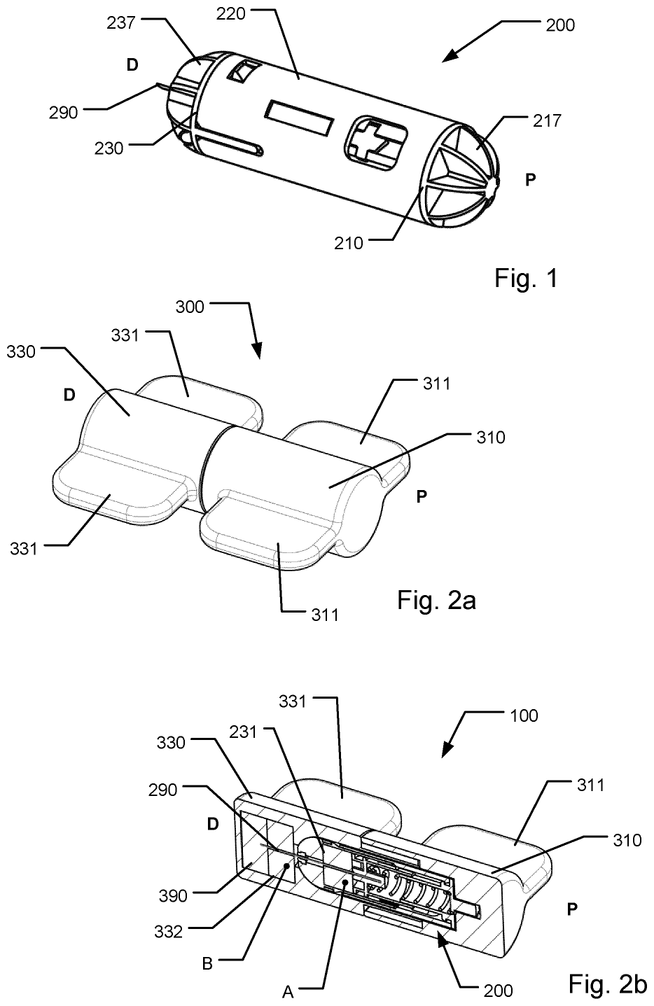

With reference to FIG. 1 a first embodiment of a drug delivery device in accordance with an aspect of the invention will be described, the embodiment being designed to provide an ingestible capsule device 200 sized and shaped to be ingested by a patient and configured for subsequently being deployed when in a target lumen of the patient so as to cause a dose of a liquid drug to be expelled through an outlet formed by an injection needle of the capsule device 200. It is to be noted that the disclosed ingestible capsule device 200, in the following referred to as “capsule 200” is only exemplary and, in accordance with the invention, may be provided in other forms having different capsule outer shapes. Also, the shown outlet may be provided as a needle with a different configuration than the one shown, may be provided as a plurality of needles, or may even be provided in a form which does not comprise an injection needle.

The disclosed embodiment relates to a capsule 200 suitable for being ingested by a patient to allow the capsule device to enter a lumen of the Gastro-Intestinal tract, such as the small intestine, and finally to eject a liquid dose of a mixed drug product at a target location either inside the lumen, or into tissue of the lumen wall surrounding the lumen. In accordance with the intended location for targeted delivery, in particular when targeted delivery is configured to occur within tissue of a lumen wall, the capsule device may include means for ensuring tissue proximity between the drug outlet and the tissue, such as a particular shape and/or mass distribution of the capsule, or by including other means for re-orienting the capsule so as to position the drug outlet in an orientation suitable for safe and consistent expelling. As such details have already been disclosed in the art, and as such details are not central to the aspects of the present invention, further details in this respect are omitted.

In the shown embodiment, the mixed drug product is intended to be prepared from at least two drug products “A” and “B”. Prior to mixing, product “A” is stored within a first reservoir 231 within capsule 200 whereas product “B” is stored exterior to the capsule 200 in a capsule accessory device 300, the capsule accessory device to be described more in depth below. After the two drug components “A” and “B” have been mixed, during a drug mixing step, the mixed drug product resides in the first reservoir 231 of capsule 200. In the shown embodiment, the drug component “A” is provided initially as a lyophilized drug substance, such as a powder, whereas the drug component “B” is a reconstitution liquid, such as a diluent. In other embodiments, the drug products “A” and “B” may be provided in other forms, such as each being initially provided as a liquid, and wherein the drug products “A” and “B” prior to drug administration are mixed for being accommodated in the first reservoir 231 for subsequent swallowing of capsule 200.

The capsule 200 shown in FIG. 1 includes a multi-part housing having an elongated shape extending along an axis, which is also referred to in the following as “the firing axis”. The elongated housing includes a cylindrical section and further include rounded end portions, i.e. a proximal end portion and a distal end portion, the latter including a drug outlet opening for the capsule 200. The drug outlet opening includes a penetrable seal, provided as an elastomeric plug 239 that is penetrable by the injection needle and which seals when not being penetrated by the injection needle. In the shown embodiment, the capsule is configured in shape and size to roughly correspond to a 00 elongated capsule. The shown multi-part housing includes a housing first portion and a housing second portion. The housing first portion is formed by proximal housing portion 210 arranged at the proximal end of the capsule 200. The housing second portion includes a generally cylindrical sleeve shaped middle housing portion 220 and a distal housing portion 230 arranged at the distal end of capsule 200. In the shown embodiment the distal housing portion 230 and the middle housing portion 220 are fixedly attached to each other by means of a permanent connection. However, the proximal housing portion 210 is mounted relative to the middle housing portion 220 so that, at least during a mixing preparation procedure for mixing separate products A and B, the proximal housing portion 210 is able to rotate relative to the middle housing portion 220 while being held axially fixed relative thereto. An injection needle 290 is movable along the firing axis in proximal and distal directions through a distal opening formed in the distal housing portion 230.

In accordance with an aspect of the present invention, the capsule 200 is configured to be accommodated within a cavity of the capsule accessory device 300 referred to above, so that the capsule accessory device 300 is used both as a packaging for storing the capsule 200 prior to use, as well as a tool for preparing the capsule 200, i.e. for providing the mixed drug product within the first reservoir 231 arranged within capsule 200. In this disclosure, the combination of capsule 200 and capsule accessory device 300 is referred to as a capsule administering assembly. A cross sectional view of a first embodiment of a capsule administering assembly 100 can be viewed in FIG. 2b. From this view, wherein the capsule administering assembly 100 is depicted in its storage state, i.e. prior to drug mixing, the drug product “B” resides outside of capsule 200, more specifically in a second reservoir 332 formed as part of accessory device 300.

For the capsule administering assembly, and more specifically to the ingestible capsule, reference is made to “a first attached state” where the ingestible capsule is coupled relative to the capsule accessory device, and “a second detached state” wherein the ingestible capsule is detached from the capsule accessory device to permit ingestion of the ingestible capsule.

Referring to FIG. 2a the capsule accessory device 300 forms two separate grippable handling components 310 and 330, both portions being generally cylindrical, which are configured so that they can be arranged and coupled relative to each other to extend along a common axis which, when capsule device 200 is accommodated within capsule accessory device 300, is coaxial with the firing axis of capsule 200. The distal handling component 330 forms a first handling component configured to engage and cooperate with the distal housing portion 230 of capsule 200. The proximal handling component 310 forms a second handling component configured to engage and cooperate with the proximal housing portion 210 of capsule 200. It is to be noted that, although the embodiments disclosed within this disclosure each comprise two separate handling components to cooperate with a single capsule device, other embodiments according to the invention only include a single handling component which cooperates with a particular design of a capsule device.

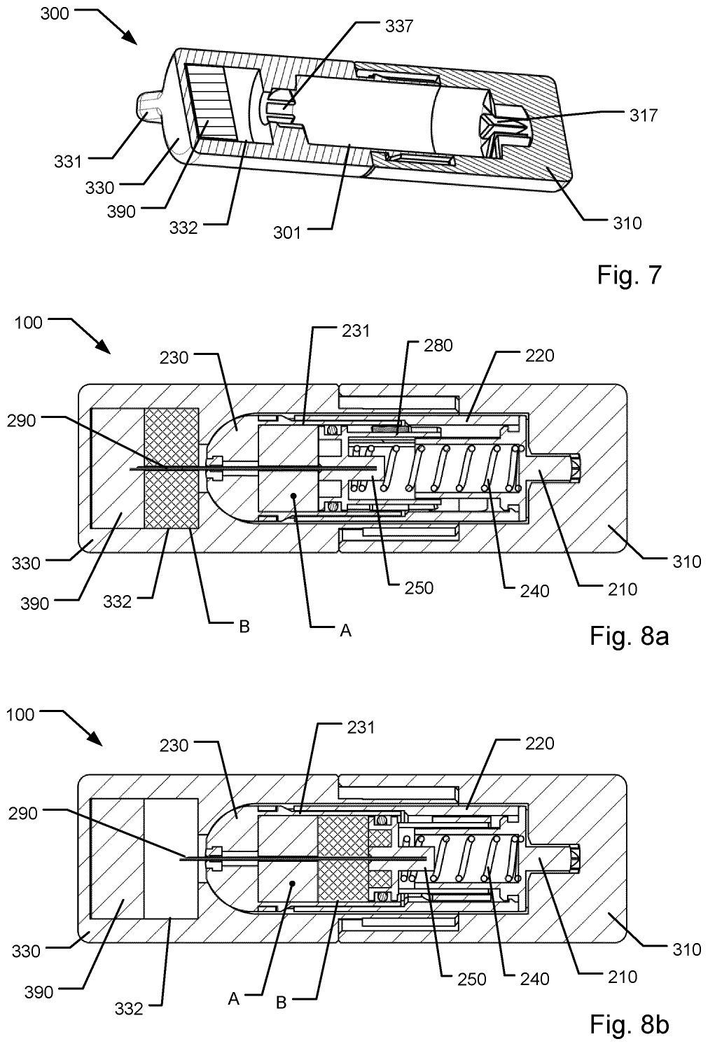

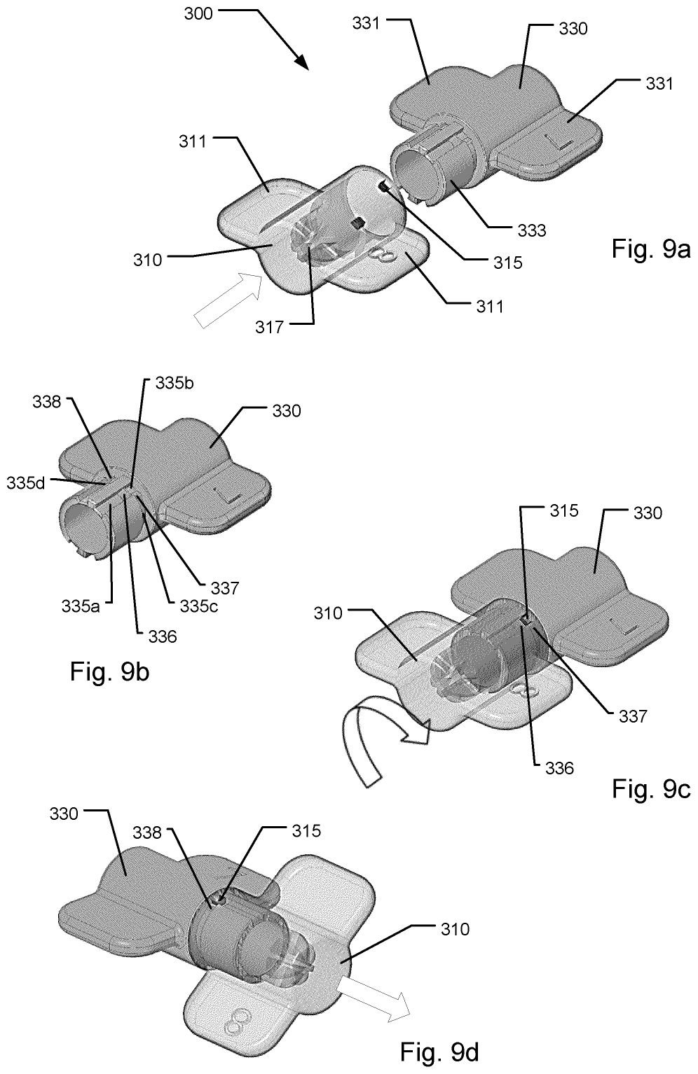

Referring also to FIGS. 7 and 9a-9d, the distal handling component 330 includes a cylindrical reduced diameter portion 333 configured to be received within a cylindrical bore of proximal handling component 310 when the two handling components are positioned relative to each other as shown in FIGS. 2a, 7, 9c and 9d. In this state, the two handling components 310 and 330 in combination form a closed volume 301 which is sized so that the capsule 200 is snugly received within volume 301, cf. also FIG. 2b. As shown in FIG. 7, the second reservoir 332 is formed in the distal handling component 330 adjoining a plug 390 made of an elastomeric material, the plug being arranged distally from the second reservoir 332. In this way the plug 390 forms one side wall of the reservoir 332.

As shown in FIG. 2a, on exterior surfaces of distal handling component 330, a pair of wings 331 are formed. Likewise, on exterior surfaces of proximal handling component 310, a pair of wings 311 are formed. The wings are shaped so that a patient, or other assisting personnel, may grip capsule accessory device 300, and then, e.g. using both hands, may initially twist the distal handling component 330 and the proximal handling component 310 relative to each other, and subsequently separate the two handling components axially from each other so at to provide access to the capsule 200.

Referring to FIGS. 1 and 7, these views reveal that distal housing portion 230 includes a non-round keyed outer surface 237 which fits in mating relationship into a non-round keyed inner surface 337 of distal handling component 330. Similarly, proximal housing portion 210 includes a non-round keyed outer surface 217 which fits in mating relationship into a non-round keyed inner surface 317 of proximal handling component 310. Hence, when the distal handling component 330 is twisted relative to the proximal handling component 310 the distal housing portion 230 is twisted relative to the proximal housing portion 210. The pairs of cooperating surfaces 237/337 and 217/317 are formed so that the capsule end portions are axially detachable relative to the respective cooperating handling components so that, after the handling components are separated axially from each other, the capsule 200 can be removed entirely from the capsule accessory device 300.

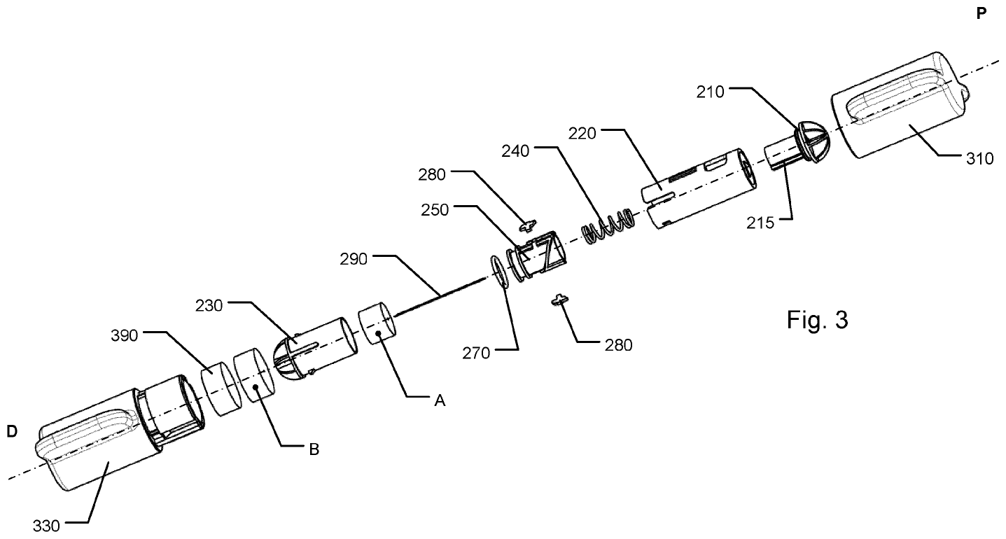

FIG. 3 shows an exploded perspective view of all the components of the first embodiment capsule administering assembly 100. It is to be noted that drug component “A”, i.e. the powder, is shown schematically as a cylindrical shaped entity corresponding to the volume of first reservoir 231 when the capsule assembly administering assembly 100 assumes the storage state. Also note that drug component “B”, i.e. the diluent, is shown schematically as a cylindrical shaped entity corresponding to the volume of the second reservoir 332 when the capsule assembly administering assembly 100 assumes the storage state.

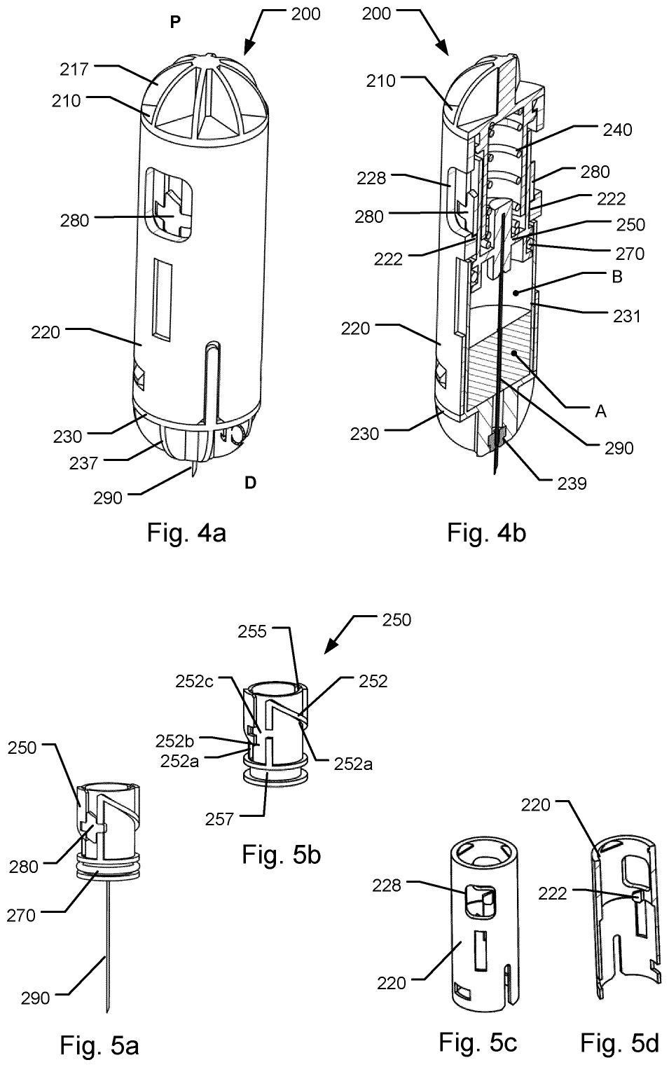

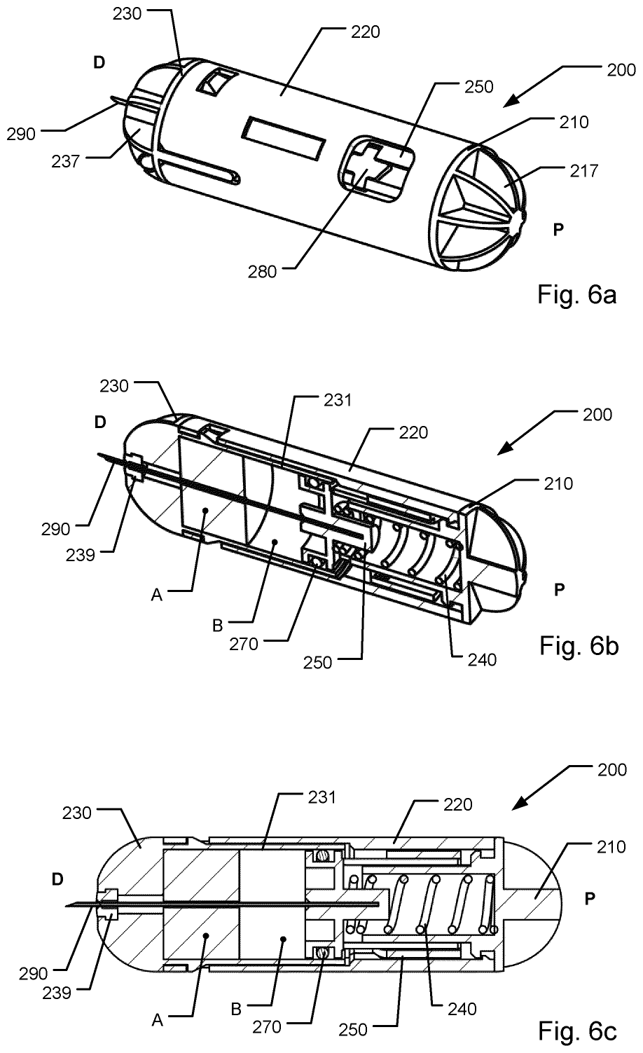

Now turning to FIGS. 4a and 4b the capsule 200 of the first embodiment will be described more in detail. The figures show the capsule in the state wherein the mixed drug product is accommodated inside first reservoir 231, and wherein the capsule is prepared ready to be ingested by a patient.

In FIG. 4b, the products “A” and “B” are schematically shown as two separate portions inside first reservoir 231. However, in real life settings, the two products will typically rapidly have become mixed or at least substantially mixed together so that a reconstituted drug product is rapidly established which fills the first reservoir 231 in a near uniform manner, however typically with an amount of air present within reservoir 231.

Inside capsule 200 the first reservoir 231 is defined by a tubular member, i.e. a cylinder, extending in the proximal direction from a distal end part of the distal housing portion 230, and so that the distal end surface of the reservoir 231 is defined by a proximally facing interior surface of distal housing portion 230. Inside the tubular member a movable needle hub 250 is arranged, the needle hub forming a piston having a ring-shaped seal 270 arranged to sealingly engage the interior wall surface of the tubular member. In the shown embodiment, during a drug mixing preparation step, the needle hub 250 is arranged to be both axially and rotationally movable relative to the middle housing portion 220 and distal housing portion 230. During drug expelling, the needle hub 250 is arranged to be axially movable relative to the middle housing portion 220 and distal housing portion 230 but prevented from moving rotationally relative to portions 220/230.

The needle hub 250 and the tubular member in combination forms a piston/cylinder assembly wherein the needle hub 250, when being moved along the firing axis in the proximal direction, provides for sucking in liquid into reservoir 231 as the volume of the reservoir is increased. Oppositely, when the needle hub 250 is moved distally, thereby reducing the volume of reservoir 231, the liquid present in the reservoir is being expelled from the first reservoir 231.

In capsule 200, a source for storing potential energy is incorporated, which in the shown embodiment is provided as a compression spring 240, arranged for being axially compressed to accumulate energy. The compression spring 240 is formed as a helical spring which is arranged coaxially with the firing axis. The proximal end of compression spring is arranged in a distal spring seat formed as an integral part of the proximal housing portion 210. The distal end of compression spring 240 is arranged in a distal spring seat formed as an integral part of the needle hub 250. Potential energy is accumulated in compression spring 240 as the needle hub 250 is moved proximally. The needle hub 250 and compression spring 240 forms part of an expelling assembly, wherein triggering of the expelling assembly is controlled by a trigger. In the shown embodiment, triggering occurs after a certain time after capsule 200 is exposed to intestinal liquid present in the small intestine.

Referring now to FIG. 5a, the needle hub 250 is generally cylindrical and part of a needle hub assembly. The needle hub assembly further includes an injection needle 290 which is fastened to the needle hub 250 so that the injection needle extends in the distal direction along the firing axis away from the needle hub. The injection needle 290 comprises a distal pointed section, a distal needle opening arranged near the distal pointed section, a proximal arranged side opening arranged in the vicinity of the needle hub 250 and an internal lumen extending between the distal needle opening and the side opening providing fluid communication between the two openings. The side opening is located so that it provides fluid communication between reservoir 231 and the lumen of the injection needle at all stages of operation of capsule 200. The needle hub assembly further includes the ring-shaped seal 270 arranged in a cylindrical channel on needle hub 250, and a pair of dissolvable firing members 280. In the state shown in FIGS. 4a and 4b, i.e. the ready-to-ingest state, the dissolvable firing members 280 provide a means of releasably retaining the needle hub 250 in a start of expelling position within first reservoir 231 to prevent it from moving distally, i.e. the dissolvable firing members 280 act as a releasable trigger.

As noted above, in accordance with aspects of the invention, the triggering systems disclosed herein are only exemplary. Other trigger systems may be used in alternative embodiments, wherein the trigger is configured to actuate in response to one or more predetermined conditions.

Referring to FIGS. 4a and 4b, when the capsule 200 assumes the ready-to-ingest state, the injection needle 290 with its distal pointed section will be substantially accommodated within the capsule 200 so that the distal needle opening is embedded within plug 239 to prevent fluid communication through the injection needle 290. Although indicated in the figures, due to their schematic nature, the injection needle appears to extend somewhat from the distal housing portion 230. However, in other embodiments, the distal pointed tip of the injection needle 290 may be either entirely embedded within plug 239, or only extending non-substantially from the capsule exterior.

As shown in FIG. 5b, a pair of symmetrically arranged radially protruding rib systems 252 protrude radially outwards from a radially outwards facing surface of the needle hub 250. Each of the pair of rib systems 252 includes a helical segment and an axial segment. Each of the rib systems 252 is configured to cooperate with a respective one of a pair of radially inwards extending protrusions 222 arranged on a radially inwards facing surface of the middle housing portion 220 (see FIG. 5d). Each protrusion 222 serves as a track follower that follows a helical segment track 252a on needle hub 250 which further leads to an axial segment track 252b, both tracks being defined by the rib system 252 of needle hub 250. When the dissolvable firing members 280 are present at location 252c, the dissolvable firing members prevent relative axial movement between the protrusions and the dissolvable firing member, thus preventing the protrusions 222 to move past location 252c in the axial segment track 252b. As shown in FIG. 5c, a pair of window openings 228 are located in housing 222 so that, with the capsule in the state shown in FIGS. 4a and 4b, the window openings 228 provides for the dissolvable firing members 280 being exposed to intestinal liquids. However, prior to ingestion, the window openings 228 may include a layer of an enteric coating designed to pass through the stomach unaltered and disintegrate at the changed pH-level in the intestine. This will cause the dissolvable firing members 280 to become exposed to the intestinal liquids with subsequent disintegration of the dissolvable firing members 280 to allow the protrusions 222 to pass the locations 252c and continue unhindered in the axial segment track 252b.

As indicated in FIG. 5b, a protrusion 255 protrudes radially inwards from a radially inwards facing surface of needle hub 250. The axial protrusion 255 is configured to be received in an axial extending groove 215 formed in the radially outwards facing surface of proximal housing portion 210, see FIG. 3. The protrusion 255 and the axial extending groove 215 serve for preventing relative rotational movement between the proximal housing portion 210 and the needle hub 250 while allowing axial relative movement between the two components to occur.

For the capsule administering assembly 100, when the capsule 200 assumes the ready-to-mix state shown in FIG. 8a (which is similar to the state shown in FIG. 2b) the capsule 200 is accommodated within the accessory capsule device 300, and the drug product “B” is present in the second reservoir 332. The drug product “A” is present in the first reservoir 231. As the needle hub 250 is positioned relatively far distally, the volume of reservoir 231 is approximately half the size compared to the state wherein the drug products “A” and “B” are mixed within reservoir 231, cf. FIG. 8b. The distal opening of the injection needle 290 is positioned so that the opening is plugged within plug 390, meaning that the two reservoirs are not in fluid communication. The proximal handling component 310 and the distal handling component 330 maintains the capsule 200 accommodated within volume 301 so that the drug product portions A and B are sealed from the external environment.

Referring to FIGS. 5a and 5d, the protrusions 222 are located along the helical segment track 252a of needle hub 250. When the subject user takes the capsule administering assembly 100 into use, the proximal handling component 310 is rotated relative to the distal handling component 330. This causes the proximal housing portion 210 and the needle hub 250 to rotate relative to the middle housing portion 220 which in turn causes the protrusions 222 to move along the rib system right to the position wherein the protrusions are rotationally located within the axial track segments 252b. During rotation, due to the interaction between the rib system 252 and the protrusion 222, the needle hub 250 is moved proximally causing the injection needle to be axially withdrawn from the plug 390 and become positioned so that there is fluid communication between the second reservoir 332 and the first reservoir 231. As the needle hub moves proximally, the size of the first reservoir 231 is increased. Hence, due to differences in hydraulic pressure, the drug product “B” is transferred from the second reservoir 332 into the first reservoir 231. As the needle hub 250 moves proximally as the handling components 310 and 330 are rotated relative to each other potential energy is accumulated in compression spring 240.

The rotation continues until the protrusions 222 reach the end of the helical part of the rib system, where after the needle hub 250 is pushed slightly distally by the compression spring 240 until the protrusions 222 arrives resting on the dissolvable firing members 280. At this point in time, the distal opening of the injection needle 290 is plugged into the plug 239, and all or a predefined portion of the drug product “B” has been transferred to reservoir 231. This causes the two drug products “A” and “B” to mix forming a mixed drug product suitable for administration. This state is shown in FIG. 8b, which is also referred to as the ready-to-ingest state for the capsule device 200.

Naturally, prior to ingestion, the capsule 200 needs to be removed from the capsule accessory device 300. In the shown embodiment, as outlined in FIGS. 9a through 9d, the distal handling component 330 and the proximal handling component 310 includes a guide system which ensures that, a predefined sequence of movement between the two is required, preventing malfunction by unintended operation. In the shown embodiment, a system of tracks and track followers are incorporated to provide this sequence control.

The proximal handling component 310 includes a pair of oppositely arranged track followers 315 protruding radially inwards into the cylindrical bore of the proximal handling component 310. A plurality of segments of axial and round-going tracks are formed on the reduced diameter portion 333 of distal handling component 330, each segments of tracks being formed to receive and guide a respective one of the track followers 315.

Referring to FIG. 9b, during assembly of the capsule administering assembly 100, i.e. after insertion of the capsule 200 into the opening formed in the reduced diameter portion of the distal handling component 330, a first axial track 335a ensures axial only movement for approaching the proximal handling component 310 relative to the distal handling component 330, cf. the arrow shown in FIG. 9a. At this stage, a one-way snap protrusion 336 ensures that the two handling components 310 and 330 cannot be axially withdrawn relative to each other. A further one-way snap protrusion 337 serves to ensure that the assembly in the state shown in FIG. 8a is maintained until the assembly is put into use.

When a deliberate rotational force is exerted between the two handling components in accordance with the arrow shown in FIG. 8c, the track followers 315 are allowed to pass the one-way snap protrusions 337 and continued rotational movement in the order of 140 deg. is performed to enable the track followers 315 to pass further one-way snap protrusions 338, cf. FIG. 9d. In this position, the capsule administering assembly 100 assumes the state shown in FIG. 8b. Axial track 335d means that further manipulation between handling components 310 and 330 can only occur by axially separating the two. When the subject user intends to swallow the capsule 200, the distal and proximal handling components 330, 310 are axially withdrawn from each other as indicated by the arrow in FIG. 9d and the capsule 200 can be entirely removed from the capsule accessory device 300. In this state the capsule is in the ready-to-ingest state.

As described above, subsequent to swallowing of the capsule device, the capsule 200 first moves through the stomach and enters the small intestine. Due to the enteric coating becomes dissolved only after passage of the stomach, the dissolvable firing members 280 are exposed to intestinal fluid. After lapse of a pre-defined time the dissolvable firing members 280 are sufficiently eroded to enable the protrusions 222 of the middle housing portion 220 to pass the locations 252c, cf. FIG. 5b. This means that the needle hub 250 is allowed to move unhindered distally, forced by the potential energy accumulated in compression spring 240, causing the injection needle 290 to be shot out of the capsule 200 and into mucosal tissue at the target location. As the needle hub 250 moves further distally, the mixed drug product accommodated in the first reservoir 231 is ejected through the lumen of the injection needle to deliver the mixed drug product for a drug depot to be formed in the mucosal tissue. After delivery of the mixed drug product, the capsule 200 is allowed to pass the alimentary canal and be subsequently excreted.

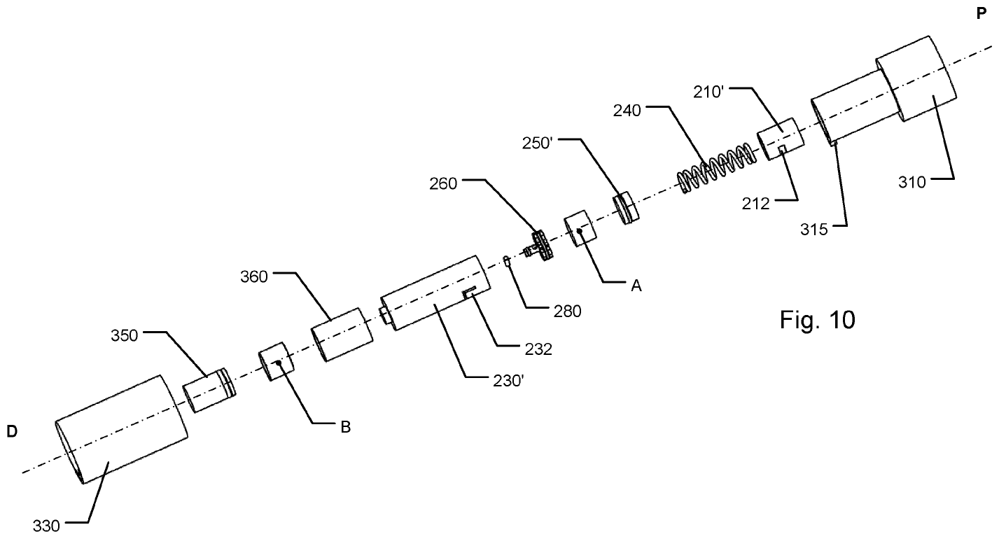

Turning now to a second embodiment of a capsule administering assembly 100′ according to the invention reference will be mainly provided to FIGS. 10 through 16b. The capsule administering assembly 100′ comprises an ingestible capsule 200′ and a capsule accessory device 300′, wherein capsule 200′ is initially accommodated within a cavity of a capsule accessory device 300′. The two disclosed embodiments share many common features and functionalities, and the description below will mainly focus on features that differ between the two embodiments.

Relative to the first embodiment, the capsule 200′ primarily differs by being configured for drug expelling into tissue of a wall of the gastrointestinal tract by means of jet injection. Existing jet injector systems for jet delivery are known in the art. A skilled person would understand how to select an appropriate jet injector that provides the correct jetting power to deliver the therapeutic substance into the lumen wall, for example from WO 2020/106,750.

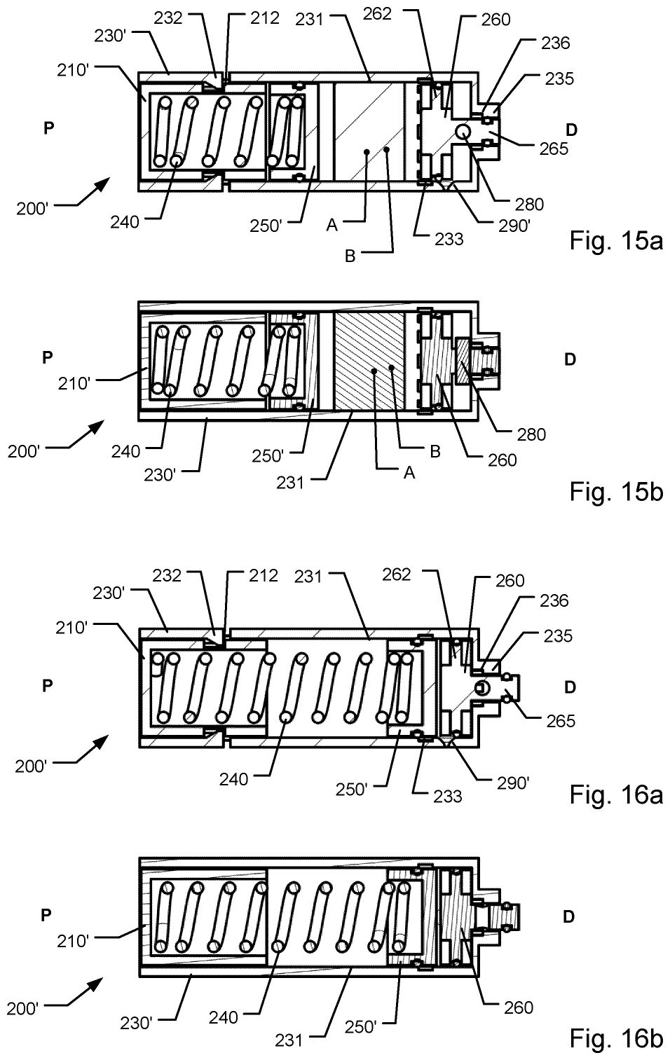

Referring mainly to FIGS. 15a and 15b, which show the capsule 200′ in the ready-to-ingest state, the capsule 200′ includes a multi-part housing having an elongated shape extending along the firing axis. The elongated housing includes a cylindrical section and end portions, i.e. a proximal end portion and a distal end portion. The shown multi-part housing is provided as a housing first portion and a housing second portion. The housing first portion is formed by distal housing portion 230′ and is provided as a generally cylindrical tubular member having a distal end wall. The distal end wall includes a reduced diameter cylindrical neck extension having a centrally located opening 235. A pair of resilient arms 232 provide for fastening means for fastening the housing second portion, i.e. proximal housing portion 210′. In the shown state the proximal housing portion 210′ fits coaxially into a proximal end portion of distal housing portion 230′. A cylindrical sleeve of proximal housing portion 210′ includes recessed areas 212 adapted to receive the pair of resilient arms 232 to mount the proximal housing portion 210′ within distal housing portion 230′.

Within capsule 200′, the generally cylindrical tubular member of distal housing portion 230′ defines a cylinder wall of a first reservoir 231. A piston 250′ having a ring-shaped seal (non-referenced) is arranged to sealingly engage the cylinder wall, in an initial state arranged approximately midways within the cylinder wall. The piston 250′ is configured for axial sliding movement in the distal direction, and optionally also in the proximal direction.

Also, within capsule 200′, and distally to piston 250′, a fluid gate control element 260 is arranged. Fluid gate control element 260 comprises a proximal portion having a large diameter flange 262 arranged for sliding engagement with the cylinder wall, i.e. with a sealing lip arranged circumferentially. Fluid gate control element 260 further comprises a reduced diameter distal section 265 arranged for sliding engagement within the centrally located opening 235 of reduced diameter cylindrical neck extension formed by distal housing portion 230′. A sealing lip is likewise arranged circumferentially on the reduced diameter distal section 265. Hence, fluid gate control element 260 is arranged slidingly within the cylinder wall of distal housing portion 230′. In the state shown in FIGS. 15a and 15b, the fluid gate control element 260 seals of the first reservoir 231 distally whereas the piston 250′ seals of the first reservoir proximally. In this state, the large diameter flange 262 of fluid gate control element 260 is arranged a short distance from the distal end wall of distal housing portion 230′.

A drug outlet in fluid communication with the first reservoir 231 is provided as a radial opening in the cylinder wall of distal housing portion 230′, positioned with a particular spacing from distal end wall of distal housing portion 230′. In the second embodiment the drug outlet is provided as a jet nozzle 290′ pointing radially outwards relative to the cylinder wall of distal housing portion 230′.

The cylinder wall of distal housing portion 230′ defines a first bypass section 233 which is arranged proximally relative to the jet nozzle 290′ of drug outlet. A further second bypass section 236 is associated with the centrally located opening 235 of the reduced diameter cylindrical neck extension, i.e. where the proximal end portion of the cylindrical neck extension intersects with the distal end wall of distal housing portion 230′.

For the capsule 200′ in the ready-to-ingest state, as shown in FIGS. 15a and 15b, fluid gate control element 260 is axially positioned so that the large diameter proximal flange 262, with its seal lip, is disposed proximally relative to jet nozzle 290′ but distally to the first bypass section 233. At the same time, the reduced diameter distal section 265, with its seal lip, is disposed distally relative to first bypass section 233 and to jet nozzle 290′. Hence, both sealing lips of fluid gate control element 260 provides a sealing function, and the mixed drug product present in the first reservoir 231 is prevented from leaking from the reservoir.

In capsule 200, a source for storing potential energy is incorporated, which in the shown embodiment is provided as a compression spring 240, arranged for being axially compressed to accumulate energy. The compression spring 240 is again formed as a helical spring which is arranged coaxially with the firing axis. The proximal end of compression spring is arranged in a distal spring seat formed as an integral part of the proximal housing portion 210′. The distal end of compression spring 240 is arranged in a distal spring seat formed as an integral part of piston 250′. Potential energy is accumulated in compression spring 240 proximal housing portion 210′ is moved distally during mounting of the proximal housing portion 210′ relative to the distal housing portion 230′.

Again, the piston 250′ and compression spring 240 forms part of an expelling assembly, wherein triggering of the expelling assembly is controlled by a trigger. In the shown embodiment, triggering occurs after a certain time after mixing of the drug components have been initiated. In the shown embodiment, this is provided by a dissolvable trigger member 280 arranged to protrude radially outwards from at opposing sides of reduced diameter distal section 26 of fluid gate control element 260 and configured for abutting contact with distal end wall of distal housing portion 230′.

In the shown embodiment of capsule 200′, fluid gate control element 260 is movable relative to distal housing portion 230′ in sequence through a first axial position, a second axial position, a third axial position and a fourth axial position. In the shown embodiment, the first axial position is the same as the third axial position, but in other embodiments, this need not be the case. The axial positions will be referred to further below.

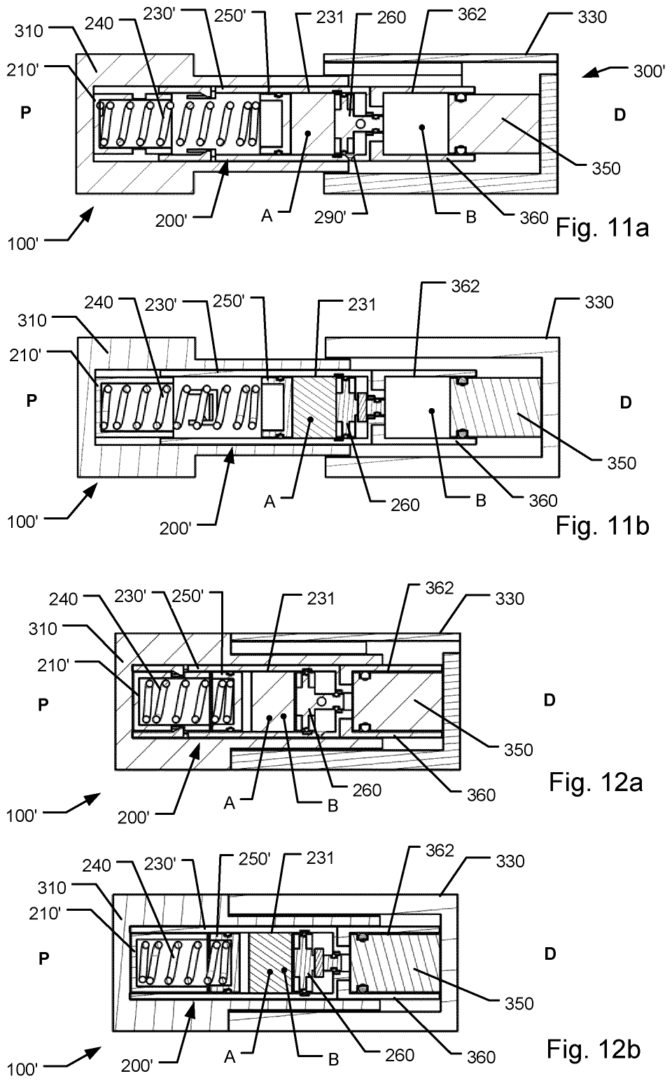

Again, in accordance with an aspect of the present invention, the capsule 200′ is configured to be accommodated within a cavity of the capsule accessory device 300′ referred to above, so that the capsule accessory device 300′ is used both as a packaging for storing the capsule 200′ prior to use, as well as a tool for preparing the capsule 200′, i.e. for providing the mixed drug product within the first reservoir 231 arranged within capsule 200′. A cross sectional view of the second embodiment of the capsule administering assembly 100 can be viewed in FIGS. 11a and 11b, wherein the capsule administering assembly 100′ is depicted in its storage state, i.e. prior to drug mixing. The drug product “B” resides outside of capsule 200′, more specifically in a second reservoir 362 formed as part of accessory device 300′.

Optionally, similar to the first embodiment, for the distal handling component 330 and the proximal handling component 310, one or more wings may be formed to aid in gripping and manipulating the handling components.

As shown in FIGS. 11a and 11b, the second reservoir 362 is formed in the distal handling component 330. In the shown embodiment of accessory device 300′, a second reservoir cylinder 360 provided as a separate component within distal handling component 330 is provided as a sleeve shaped element having radially outwards and radially inwards dimensions comparable to the generally cylindrical tubular member of distal housing portion 230′. In the attached state shown in FIGS. 11a through 12b, the second reservoir cylinder 360 is mounted sealingly relative to distal housing portion 230′ of capsule 200′.

As seen in the drawings, reservoir element 360 mates with the distal end of distal housing portion of capsule 200′, when the components 200′ and 360 are coupled to each other, fluid communication between the interior of reservoir element 360 and the first reservoir 231 arranged within capsule 200′ is possible, however subject to the state of the fluid gate control element 260.

A second reservoir piston member 350 fits within an interior bore of second reservoir cylinder 360. Second reservoir piston member 350 is initially located at a distal end of second reservoir cylinder 360 and provides a distal end surface arranged for cooperation with a proximally facing surface formed in the interior of distal handling component 330. When distal handling component 330 is forced proximally relative to the capsule 200′ the second reservoir piston member 350 is moved proximally inside second reservoir cylinder 360 thereby reducing the volume of the second reservoir 362.

For capsule accessory device 300′, the operational sequence of the second embodiment is somewhat different from the first embodiment capsule accessory device 300 described above, and the guide system is different. Although not shown in details in the figures of this disclosure, the proximal handling component 310 and the distal handling component 330 include cooperating sets of tracks and track followers to define a predefined sequence of relative movements that the two handling components are required to follow. For the shown embodiment, the guide system is configured to define an axial segment, followed by a circumferential segment and finalized by an axial segment. One track follower 315 extending radially outwards from a reduced diameter section of proximal handling component 310 is visible in FIG. 10, the track follower being configured for cooperating engagement with a track system formed at or in a radially inwards facing surface of distal handling component 330.

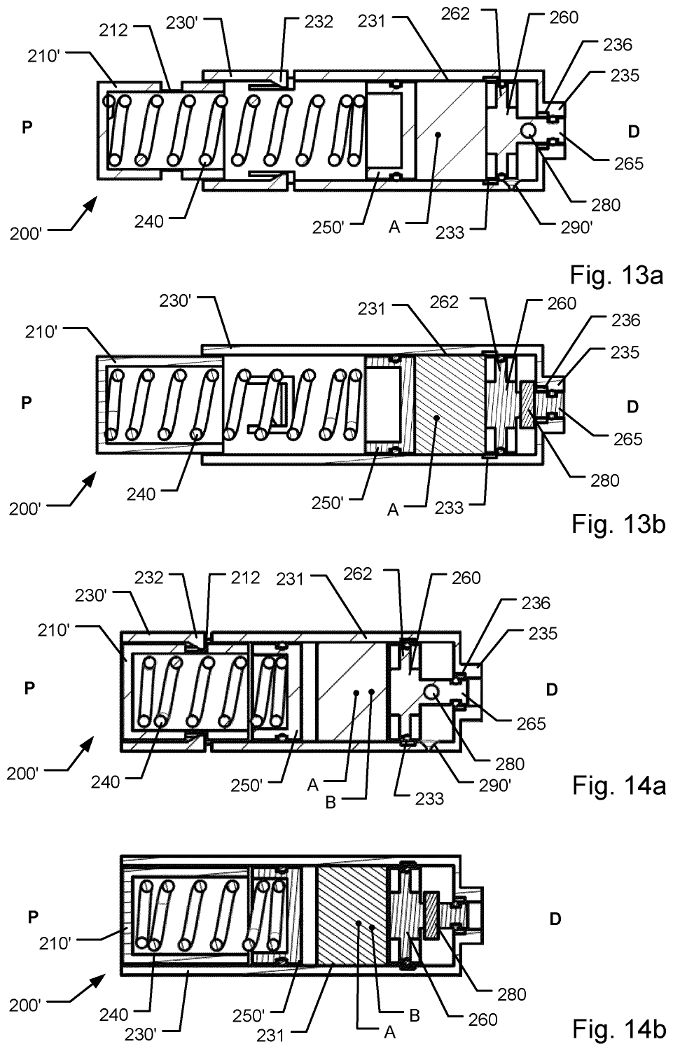

Referring to the view shown in FIGS. 11a and 11b, depicting the capsule administering assembly 100′ wherein the capsule 200′ assumes the ready-to-mix state shown in FIGS. 13a and 13b, the capsule 200′ is accommodated within the accessory capsule device 300′, and the drug product “B” is present in the second reservoir 362. The drug product “A” is present in the first reservoir 231. The compression spring 240 is either non-compressed or alternatively only slightly compressed as the proximal housing portion 210′ has not yet been axially inserted into the distal housing portion 230′. The proximal handling component 310 and distal handling component 330 are situated axially in a maximally extended position allowing the second reservoir to obtain a large volume and the compression spring to extend non-compressed or only slightly compressed. The fluid gate control element 260 is disposed in the first axial position where the seal lip of the reduced diameter distal section 265 seals of the second reservoir 362 and where the seal lip of the large diameter flange 262 seals of the first reservoir 231.

Referring to the views shown in FIGS. 12a and 12b, depicting the capsule administering assembly 100′ wherein the capsule 200′ assumes the mixed state shown in FIGS. 14a and 14b, the proximal handling component 310 and distal handling component 330 have been manually axially moved towards each other i.e. by way of a telescoping movement, so that the combined length assumes a minimum axial extension. The force exerted manually to do this has moved the second reservoir piston member 350 fully into the second reservoir 362 so as to reduce its volume to a minimum. The elevated fluid pressure inside the second reservoir 362 has forced the fluid gate control element 260 into its second axial position. In this position, the large diameter proximal flange 262, with its seal lip, is positioned axially at the first bypass section 233 whereas the reduced diameter distal section 265, with its seal lip, is positioned axially at the second bypass section 236. Hence, both these seal lips of fluid gate control element 260 are open and the fluid pressure in the second reservoir has caused the drug product “B” to flow to the first reservoir 231 where the mixed drug product is rapidly established. The axial force provided onto the handling components has at the same time caused the proximal housing portion 210′ to be moved fully axially into the opening of the distal housing portion 230′ and a permanent mounting of these two components have been made by means of features 212 and 232. Hence, the compression spring has been maximally compressed thereby accumulating potential energy in compression spring 240. An amount of liquid drug product “B” present between the lip seals of fluid gate control element 260 will initiate slowly eroding the dissolvable trigger member 280.

Subsequent to relative rotation between handling components 310 and 330, the two handling components can be axially extracted from each other and the capsule 200′ can be removed from the capsule accessory device 300′. For capsule 200′ this state is depicted in FIGS. 15a and 15b. Due to the load exerted by compression spring 240 onto piston 250′, the fluid gate control element 260 has been moved slightly distally into its third axial position where the dissolvable trigger member 280 abuts the distal end wall of distal housing portion 230′ and the compression spring 240 is prevented from expanding further. The large diameter proximal flange 262, with its seal lip, is positioned axially between the first bypass section 233 and the jet nozzle 290′. The capsule 200′ now assumes its ready-to-ingest state.

Subsequent to a patient ingests capsule 200′, the capsule travels through the stomach region and into the small intestine. The dissolvable trigger member 280 has been designed to erode gradually with a controlled triggering in accordance with the desired target location within the small intestine for drug expelling. The capsule 200′ now assumes a state wherein the dissolvable trigger member 280 has been sufficiently eroded for the potential energy stored in compression spring 240 is released. Hence, the hydraulic pressure of the mixed drug product has caused the fluid gate control element 260 to move distally into its fourth and final axial position wherein the large diameter proximal flange 262, with its seal lip, is positioned axially distally relative to the jet nozzle 290′. Hence, the fluid elevated hydraulic pressure present in the first reservoir 231 causes a jet stream of liquid to be formed downstream from the jet nozzle 290′ causing a high velocity jet formation sufficient to penetrate the GI mucosal lining located at a wall section of the intestinal wall and resulting in a depot formation of the mixed drug inside the mucosal lining. In the state wherein capsule 200′ assumes the end of expelling state, as depicted in FIGS. 16a and 16b, the piston 250 has been moved fully distally and all the useable amount of the mixed drug product has been expelled through the jet nozzle 290′. After delivery of the mixed drug product, the capsule 200′ is allowed to pass the alimentary canal and be subsequently excreted.

Although the above description of exemplary embodiments mainly concern ingestible capsules for delivery in the small intestine, the present invention generally finds utility in capsule devices for lumen insertion in general, wherein a capsule device is positioned into a body lumen for delivery of a mixed drug product. Non-limiting examples of capsule devices include capsule devices for delivery in the stomach of a mixed drug product. Drug delivery may be configured to occur into the tissue wall of a body lumen, such as a lumen of the stomach. Drug delivery may be performed using a delivery member, such as a needle, via a jet stream of liquid to provide liquid jet penetration into the mucosal lining or via spraying inside the lumen, i.e. for systemic release.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A capsule administering assembly comprising an ingestible capsule for delivery of a mixed drug product to a subject user, and a capsule accessory device, wherein the ingestible capsule is operable from a first attached state where the ingestible capsule, is coupled relative to the capsule accessory device, and a second detached state wherein the ingestible capsule is detached from the capsule accessory device, to permit ingestion of the ingestible capsule,
wherein the capsule administering assembly, comprises a first reservoir holding a first drug component and a second reservoir separate from the first reservoir, the second reservoir holding a second drug component, and wherein the capsule administering assembly further comprises fluid communication means structure for establishing fluid communication between the first reservoir and the second reservoir, and
wherein the ingestible capsule comprises:
a capsule housing sized to pass through the gastrointestinal tract of a patient, the capsule housing comprising a capsule housing first portion,
the first reservoir arranged within the capsule housing,
a drug outlet, and
an expelling assembly comprising a movable wall associated with the first reservoir, the movable wall being movable relative to the capsule housing first portion to reduce the volume of the first reservoir thereby expelling drug from the first reservoir through the drug outlet,
wherein the capsule accessory device comprises a first handling component configured for movement relative to the capsule housing first portion,
wherein when the ingestible capsule assumes the first attached state, the first handling component is moveable relative to the capsule housing first portion from a first position to a second position, wherein the capsule administering assembly is configured to transfer the second drug component from the second reservoir via the fluid communication structure to the first reservoir upon movement of the first handling component from the first position to the second position, thereby forming said mixed drug product accommodated in the first reservoir.

2. A capsule administering assembly as in claim 1, wherein the capsule accessory device comprises said second reservoir.

3. A capsule administering assembly as in claim 1, wherein the capsule administering assembly, is configured to increase the volume of the first reservoir upon movement of the first handling component from the first position to the second position thereby forcing the second drug component from the second reservoir to the first reservoir.

4. A capsule administering assembly as in claim 1, wherein the capsule administering assembly, is configured to reduce the volume of the second reservoir upon movement of the first handling component from the first position to the second position thereby forcing the second drug component from the second reservoir to the first reservoir.

5. A capsule administering assembly as in claim 1, wherein the capsule accessory device further comprises a second handling component being movably arranged relative to the first handling component, wherein when the ingestible capsule assumes the attached state, the first handling component moves relative to the second handling component as the first handling component moves relative to the capsule housing first portion from said first position to said second position.

6. A capsule administering assembly as in claim 5, wherein the first handling component is configured for rotationally moving relative to the capsule housing first portion from the first position to the second position.

7. A capsule administering assembly as in claim 6, wherein the capsule housing further comprises a capsule housing second portion rotationally movable relative to the capsule housing first portion.

8. A capsule administering assembly as in claim 7, wherein, as the first handling component moves relative to the capsule housing first portion from said first position to said second position, the first handling component couples rotationally with the capsule housing second portion and the second handling component couples rotationally with the capsule housing first portion.

9. A capsule administering assembly as in claim 7, wherein the movable wall defines comprises a displaceable piston, and wherein the ingestible capsule, comprises a mechanism coupling the displaceable piston, the capsule housing first portion and the capsule housing second portion with each other, wherein said mechanism incorporates a helical guide structure configured to displace the piston to increase the volume of the first reservoir as the capsule housing second portion rotates relative to the capsule housing first portion.

10. A capsule administering assembly as in claim 5, wherein, when the ingestible capsule, assumes the attached state, the first handling component and the second handling component in combination define a cavity, wherein the ingestible capsule, is partly of fully received in the cavity.

11. A capsule administering assembly as in claim 5, wherein the capsule administering assembly comprises a guide system enabling the ingestible capsule to be detached relative to the capsule accessory device, subsequently to the first handling component being moved relative to the capsule housing first portion from the first position to the second position, the guide system preventing detachment relative to the capsule accessory device prior to the first handling component being moved to the second position.

12. A capsule administering assembly as in claim 1, wherein the first handling component is configured for axially moving relative to the capsule housing first portion from the first position to the second position.

13. A capsule administering assembly as in claim 1, wherein the fluid communication structure comprises a fluid gate operable between a closed state wherein the fluid gate separates the first drug component and the second drug component from each other, and an open state wherein the fluid gate enables fluid flow from the second reservoir to the first reservoir.

14. A capsule administering assembly as in claim 1, wherein one of the first drug component and the second drug component is a powder, and wherein the other of the first drug component and the second drug component is a liquid.

15. A capsule administering assembly as in claim 1, wherein the first drug component is a liquid and the second drug component is a liquid.
